(19) United States
(12) Patent Application Publication
(10) Pub. No.: US 2018/0200432 A1
(43) Pub. Date: Jul. 19, 2018

(54) DEVICE FOR ADMINISTERING DRUG SOLUTION

(71) Applicant: Terumo Kabushiki Kaisha, Tokyo (JP)

(72) Inventors: Mitsutoshi Yaegashi, Aomori-shi (JP); Yoshihisa Sugawara, Oiso-machi (JP)

(73) Assignee: Terumo Kabushiki Kaisha, Tokyo (JP)

(21) Appl. No.: 15/919,316

(22) Filed: Mar. 13, 2018

Related U.S. Application Data
(63) Continuation of application No. PCT/JP2016/071271, filed on Jul. 20, 2016.

(30) Foreign Application Priority Data
Sep. 14, 2015 (JP) 2015-181054

(51) Int. Cl. A61M 5/168; G01F 1/684; A61M 5/142

(52) U.S. Cl. CPC A61M 5/16804; G01F 1/6847; G01F 15/005; A61M 5/14244; A61M 5/16886

(57) ABSTRACT

A device for administering a drug solution includes: a pumping apparatus configured to deliver a drug solution held in a reservoir to a live body; a flow rate sensing apparatus configured to determine a flow rate of the solution delivered by the pumping apparatus, the flow rate sensing apparatus comprising: a heat-generating element disposed in a conduit line in which the drug solution is delivered; a first temperature sensor disposed in the conduit line upstream of the heat-generating element; a second temperature sensor disposed in the conduit line downstream of the heat-generating element; and an processor configured to calculate the flow rate in the conduit line based on a difference between a temperature detected by the first temperature sensing element and a temperature detected by the second temperature sensing element; and a controller configured to control a drive state of the pumping apparatus based on the determined flow rate.

### CROSS-REFERENCE TO RELATED APPLICATIONS

The present application is a bypass continuation of PCT Application No. PCT/JP2016/071271, filed on Jul. 20, 2016, which claims priority to Japanese Application No. 2015-181054, filed on Sep. 14, 2015, the contents of both of which are hereby incorporated by reference in their entireties.

### BACKGROUND

The present disclosure relates to a device for administering a drug-solution, for example, a portable drug-solution administration device for administering insulin into a body.

A portable insulin administration device has been developed that automatically administers insulin, which is a drug solution for controlling blood glucose, subcutaneously to a diabetic patient. A small-sized pump is built into the portable insulin administration device, and a drug solution (insulin) stored in a reservoir is administered to the patient by driving the pump.

When administering a drug solution such as insulin, it is necessary to administer the drug solution at a low flow rate with high accuracy. For accurate administration at a low flow rate, a conventional insulin administration device adopting a syringe pump system in which a stepping motor and a decelerator are combined has been typically used.

JP 2002-126092 A describes an example of a drug transport device applicable to an insulin administration device, and an example of using a piezoelectric pump using a piezoelectric element is described.

### SUMMARY

A conventional syringe pump system with a stepping motor and a decelerator combined has disadvantages of being difficult to reduce in size and of having a high operating noise. Thus, the system is not suitable for a portable type drug-solution administration device that is small in size. For this reason, application of a miniaturized pump such as a piezoelectric pump as described in JP 2002-126092 A has been studied. However, a miniaturized pump such as a piezoelectric pump has a problem that the accuracy thereof at a low flow rate is insufficient.

One object of certain embodiments describe herein is to provide a drug-solution administration device suitable for miniaturization, and that enables administration of a drug solution with high accuracy at a low flow rate.

According to one embodiment, a drug-solution administration device includes a pumping apparatus for feeding a drug solution held in a reservoir and administering the drug solution to a live body, a flow rate sensing apparatus for detecting a flow rate of the solution fed by the pumping apparatus, and a drive unit for controlling a drive state of the pumping apparatus on the basis of the flow rate detected by a flow rate sensing apparatus.

The flow rate sensing apparatus includes a heat-generating element disposed in a conduit line for sending a drug solution, a first temperature sensing element disposed in a conduit line on the upstream side of the heat-generating element, a second temperature sensing element disposed in a conduit line on the downstream side of the heat-generating element, and an processor for calculating the flow rate in the conduit line based on the difference between temperatures detected by the first and second temperature sensing elements.

According to certain embodiments of the drug-solution administration device describe herein, even when the flow rate of the drug solution produced by driving of the pumping apparatus is low, the flow rate sensing apparatus can detect the accurate mount of drug solution administered, and the drive unit can control the drive state of the pumping apparatus with high accuracy based on the detected accurate amount of drug solution administered. Therefore, according to certain embodiments of the drug-solution administration device described herein, a drug solution can be administered at a low flow rate with high accuracy.

### BRIEF DESCRIPTION OF THE DRAWINGS

FIG. 1 is a perspective view illustrating an internal configuration example of a drug-solution administration device according to an embodiment.

FIG. 2 is a cross-sectional view illustrating a configuration example of a flow rate sensing apparatus provided in the drug-solution administration device according to an embodiment.

FIG. 3 is a circuit diagram illustrating a circuit configuration example connected to a temperature sensing element of the flow rate sensing apparatus according to an embodiment.

FIG. 4 is a circuit diagram illustrating a configuration example of a drive circuit of a heat-generating element of the flow rate sensing apparatus according to an embodiment.

FIG. 5 is a characteristic diagram illustrating an example of a flow rate of a drug solution by a pumping apparatus according to an embodiment.

FIG. 6 is a characteristic diagram illustrating an example of a flow rate sensing state according to an embodiment.

FIG. 7 is a characteristic diagram illustrating an example of the relationship between detected voltage and the flow rate according to an embodiment.

FIG. 8 is a characteristic diagram illustrating an example of the relationship between the detected voltage and the temperature according to an embodiment.

FIG. 9 is a characteristic diagram illustrating in detail an example of the relationship between the detected voltage and the temperature according to an embodiment.

FIG. 10 is a timing chart illustrating a driving example of the pumping apparatus according to an embodiment.

FIG. 11 is a characteristic diagram illustrating a control example at a specific temperature according to an embodiment.

FIG. 12 is a cross-sectional view illustrating a configuration example of the flow rate sensing apparatus in a modification example according to an embodiment.

FIG. 13 is a cross-sectional view taken along line A-A in FIG. 12.

### DETAILED DESCRIPTION

Hereinafter, a drug-solution administration device according to embodiments of the present invention will be described with reference to the accompanying drawings.

### 1. Overall Configuration of Drug-Solution Administration Device

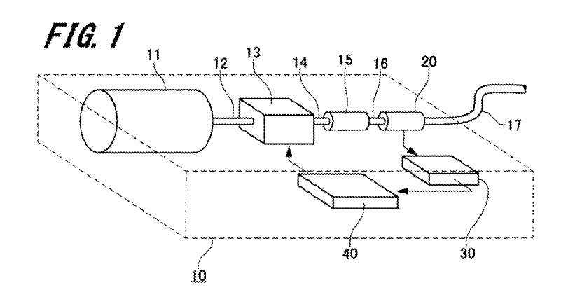

FIG. 1 shows an internal configuration of a drug-solution administration device 10. The drug-solution administration device 10 is housed in a compact casing as a portable type and used as an insulin administration device.

As shown in FIG. 1, the drug-solution administration device 10 includes a reservoir 11 for storing a drug solution (here, insulin). A pumping apparatus 13 is connected to the reservoir 11 via a conduit line 12.

As the pumping apparatus 13, a piezoelectric pump is used. The piezoelectric pump vibrates a diaphragm disposed inside by using a piezoelectric element and delivers the drug solution held in the reservoir 11 to a conduit line 14. A valve which keeps the liquid feeding direction of the drug solution constant is built in the pumping apparatus 13.

When the pumping apparatus 13 does not contain a valve, an on-off valve may be disposed in the conduit line 12 between the reservoir 11 and the pumping apparatus 13 to be opened and closed in conjunction with the vibration of the diaphragm in the pumping apparatus 13.

A microneedle valve 15 is connected to the conduit line 14 to which the drug solution is sent from the pumping apparatus 13. The liquid feeding state for the drug solution from the pumping apparatus 13 is pulsative, and the microneedle valve 15 acts to suppress this pulsation. A flow rate sensing apparatus 20 is connected to a conduit line 16 to which a drug solution whose pulsation is suppressed by the microneedle valve 15 is delivered. It should be noted that the microneedle valve 15 may not be disposed between the conduit lines 14 and 16.

A conduit line 17 drawn out to the outside of the drug-solution administration device 10 is connected to the flow rate sensing apparatus 20, and the drug solution is injected into a live body via the conduit line 17. The configuration for injecting the drug solution into the body will be omitted.

Respective elements (a first temperature sensing element 22, heat-generating element 23, second temperature sensing element 24 to be described later) incorporated in the flow rate sensing apparatus 20 are connected to an processor 30. The processor 30 detects the flow rate of the drug solution passing through the flow rate sensing apparatus 20 based on the state of each element. Information on the flow rate detected by the processor 30 is supplied to a drive unit 40 (also referred to as a “controller”).

The drive unit 40 determines the necessary amount of the drug solution administered according to the operation mode of the drug-solution administration device 10, and controls the drive state of the pumping apparatus 13. At this time, the drive state of the pumping apparatus 13 is corrected and the amount of the drug solution administered is controlled to an accurate state based on the information on the flow rate detected by the processor 30. When the drive unit 40 controls the pumping apparatus 13 formed of a piezoelectric pump, the liquid feeding amount is controlled by setting the driving voltage and the driving frequency.

### 2. Configuration of Flow Rate Sensing Apparatus

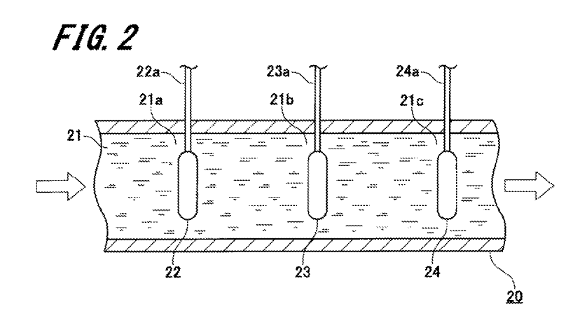

FIG. 2 shows the configuration of the flow rate sensing apparatus 20.

The flow rate sensing apparatus 20 includes a conduit line 21 in which the first temperature sensing element 22, the heat-generating element 23, and the second temperature sensing element 24 are arranged.

The first temperature sensing element 22, the heat-generating element 23, and the second temperature sensing element 24 are arranged from the upstream side of the conduit line 21 to be substantially equally spaced. That is, the first temperature sensing element 22 is disposed at the most upstream side position 21a inside the conduit line 21. Then, the heat-generating element 23 is disposed at a position 21b that is separated by a predetermined distance from the position 21a where the first temperature sensing element 22 is disposed. Further, the second temperature sensing element 24 is disposed at a position 21c that is separated by a predetermined distance from the position 21b where the heat-generating element 23 is disposed. As the first temperature sensing element 22 and the second temperature sensing element 24, thermistors whose resistance values vary with temperature are used, for example. Also for the heat-generating element 23, a thermistor for a heater is used. In the following description, the first temperature sensing element 22 and the second temperature sensing element 24 are referred to as an upstream-side temperature sensing element and a downstream-side temperature sensing element.

Then, the processor 30 connected to the flow rate sensing apparatus 20 drives the heat-generating element 23 to generate heat and detects the flow rate inside the conduit line 21 in accordance with the temperatures detected by the upstream-side temperature sensing element 22 and the downstream-side temperature sensing element 24.

The details of the principle of detection of the flow rate from the temperatures detected by the upstream-side temperature sensing element 22 and the downstream-side temperature sensing element 24 of the flow rate sensing apparatus 20 will be described later, but this can be explained briefly as follows. For example, when the heat-generating element 23 heats the drug solution in the conduit line 21 in a state where there is no flow rate in the conduit line 21, the temperatures detected by two temperature sensing elements 22 and 24 having the same distance from the heat-generating element 23 are equal. On the other hand, when some flow rate is generated in the conduit line 21, the temperatures detected by the two temperature sensing elements 22 and 24 are different from each other. Utilizing this principle, the flow rate sensing apparatus 20 detects the flow rate from the difference between temperatures detected by the two temperature sensing elements 22 and 24.

### 3. Circuit Connected to Each Element

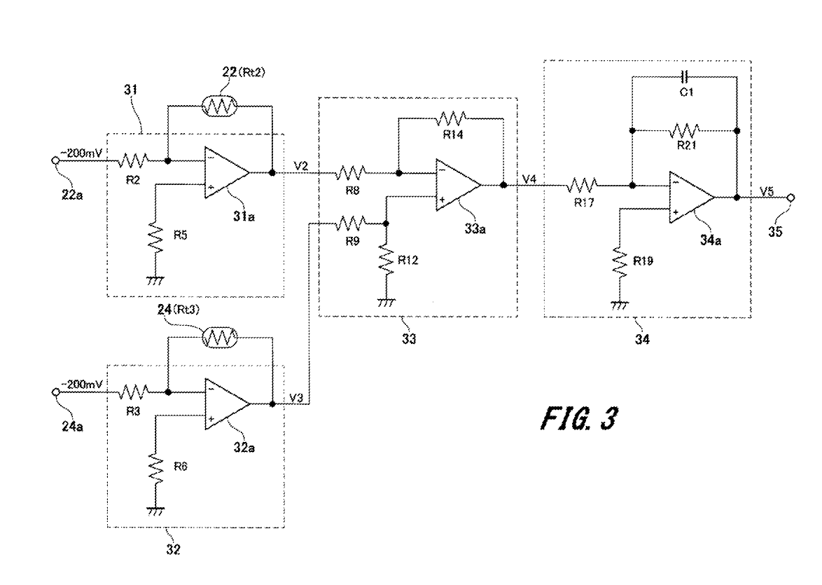

FIG. 3 is a circuit example of the processor 30 connected to the upstream-side temperature sensing element 22 and the downstream-side temperature sensing element 24. The resistance value Rt2 of the upstream-side temperature sensing element 22 is converted into a voltage value by a first voltage conversion circuit 31 and the resistance value Rt3 of the downstream-side temperature sensing element 24 is converted into a voltage value by a second voltage conversion circuit 32.

With regard to the configuration of the first voltage conversion circuit 31, the upstream-side temperature sensing element 22 formed of a thermistor is connected between the inverting input terminal (−) and the output terminal of an operational amplifier 31a constituting the first voltage conversion circuit 31. A predetermined voltage (for example, −200 mV) is applied from a terminal 22a to the inverting input terminal (−) of the operational amplifier 31a via a resistor R2.

The non-inverting input terminal (+) of the operational amplifier 31a is connected to the ground via a resistor R5. The voltage V2 obtained at the output terminal of the operational amplifier 31a has a voltage value proportional to the resistance value Rt2 of the upstream-side temperature sensing element 22.

The configuration of the second voltage conversion circuit 32 is the same as that of the first voltage conversion circuit 31. That is, the downstream-side temperature sensing element 24 formed of a thermistor is connected between the inverting input terminal (−) and the output terminal of an operational amplifier 32a constituting the second voltage conversion circuit 32. A predetermined voltage (for example, −200 mV) is applied to the inverting input terminal (−) of the operational amplifier 32a from a terminal 24a via a resistor R3.

The non-inverting input terminal (+) of the operational amplifier 32a is connected to the ground through a resistor R6. The voltage V3 obtained at the output terminal of the operational amplifier 32a has a voltage value proportional to the resistance value Rt3 of the downstream-side temperature sensing element 24.

The voltage V2 obtained by the first voltage conversion circuit 31 and the voltage V3 obtained by the second voltage conversion circuit 32 are input to a differential amplifier circuit 33 so that a differential voltage V4 between both the voltages is acquired. That is, the voltage V2 obtained by the first voltage conversion circuit 31 is applied to the inverting input terminal (−) of an operational amplifier 33a constituting the differential amplifier circuit 33 via a resistor R8. The voltage V3 obtained by the second voltage conversion circuit 32 is applied to the non-inverting input terminal (+) of the operational amplifier 33a via a resistor R9.

The connection point between the resistor R9 and the non-inverting input terminal (+) of the operational amplifier 33a is connected to the ground via a resistor R12, and the inverting input terminal (−) and the output terminal of the operational amplifier 33a are connected by a resistor R14. The amplification factor of the output voltage V4 of the differential amplifier circuit 33 is determined according to the resistance value of each resistor. A detailed example of the amplification factor will be described later.

The differential voltage V4 obtained by the differential amplifier circuit 33 is applied to a low-pass filter 34, and the voltage V5 from which the high frequency component which is noise has been removed is output to an output terminal 35. Specifically, the low-pass filter 34 is composed of an operational amplifier 34a, resistors R17, R19, and R21, and a capacitor C1 connected to the operational amplifier 34a. In the low-pass filter 34, the voltage V5 from which high-frequency noise has been removed can be obtained by the action of the resistor R21 and the capacitor C1.

The processor 30 calculates the average of the voltage V5 to obtain the flow rate.

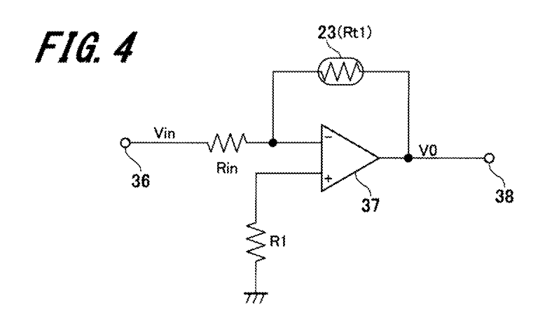

FIG. 4 is a diagram showing an example of a circuit connected to the heat-generating element 23. The circuit shown in FIG. 4 is also provided in the processor 30. The voltage Vin applied to a terminal 36 is impressed to the inverting input terminal (−) of an operational amplifier 37 via a resistor Rin. The non-inverting input terminal (+) of the operational amplifier 37 is connected to the ground through a resistor R1.

Then, the heat-generating element 23 is connected between the inverting input terminal (−) and the output terminal of the operational amplifier 37. The voltage V0 output to the output terminal of the operational amplifier 37 is obtained at a terminal 38. The voltage V0 obtained at the terminal 38 is used for detecting the liquid temperature at the position of the heat-generating element 23 during a heat dissipation period (idle period of heat generation) to be described later. In the description of the operation to be described later, the resistance value of the heat-generating element 23 is referred to as Rt1.

### 4. Example of Feeding State by Pumping Apparatus

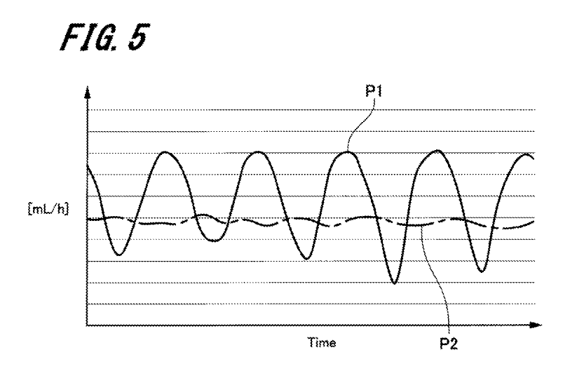

FIG. 5 shows an example of a liquid feeding state by the pumping apparatus 13. In FIG. 5, the vertical axis represents the flow rate and the horizontal axis represents time. A characteristic P1 shown in FIG. 5 indicates the fluctuation in the flow rate of the pumping apparatus 13 alone. In the pumping apparatus 13 using a piezoelectric pump, the characteristic P1 of the flow rate of a single unit fluctuates by about ±15% with respect to the flow rate desired to be set originally due to the influence of temperature or the like.

A characteristic P2 shown in FIG. 5 indicates the fluctuation of the flow rate at the output portion of the microneedle valve 15. By providing the microneedle valve 15, the fluctuation amount of the flow rate can be reduced as illustrated.

### 5. Principle of Detection of Flow Rate by Flow Rate Sensing Apparatus

Next, the principle of detection of the flow rate using each element arranged in the flow rate sensing apparatus 20 will be described.

For example, when the heat-generating element 23 heats the drug solution in the conduit line 21 in a state in which there is no flow rate inside the conduit line 21 of the flow rate sensing apparatus 20, the temperatures detected by the two temperature sensing elements 22 and 24 having the same distance from the heat-generating element 23 are equal to each other. On the other hand, when there is some flow rate in the conduit line 21, the temperatures detected by the two temperature sensing elements 22 and 24 are different from each other.

Here, suppose that the resistance value of the upstream-side temperature sensing element 22 made of a thermistor whose resistance value varies with temperature is Rt2, the resistance value of the downstream-side temperature sensing element 24 is Rt3, and the constant current I is supplied to each of the temperature sensing elements 22 and 24, the output voltages V2 and V3 of the first voltage conversion circuit 31 and the second voltage conversion circuit 32 are expressed by the following equations (1) and (2). The current I is selected in the range of 0.02 mA to 0.05 mA, for example. In this case, the current I is 0.02 mA.

V2=I·Rt2 (1)

V3=I·Rt3 (2)

Further, if the amplification factor of the output voltage V4 of the differential amplifier circuit 33 to which the voltages V2 and V3 are input is G, the voltage V4 is expressed by the following equation (3). The equation (3) can also be expressed as equation (4). The amplification factor G is 10 times, for example.

V4=G(V3−V2) (3)

V4=G·I(Rt3−Rt2) (4)

The amplification factor G is set by the ratio of the resistance values of the resistors R8 and R14. Namely, G=R14/R8. However, it is assumed that the resistors R8 and R9 have the same resistance value and the resistors R12 and R14 have the same resistance value.

With respect to the temperature T [° C.] within a specific temperature range, the resistance value Rt of the thermistor is expressed by the following equation (5).

Rt=−a·T+b (5)

Here, a and b are positive constants. In this example, a negative temperature coefficient (NTC) thermistor having a characteristic in which the resistance value decreases with increase in temperature is used as the thermistor.

Therefore, the voltage V4 can be expressed as the following equation (6) by substituting the equation (5) into the equation (4). In the equation (6), T2 is the temperature of the upstream-side temperature sensing element 22, and T3 is the temperature of the downstream-side temperature sensing element 24.

V4=G·I·a(T3−T2) (6)

From this equation (6), it can be seen that the output voltage V4 of the differential amplifier circuit 33 is proportional to the temperature difference between the upstream-side temperature sensing element 22 and the downstream-side temperature sensing element 24. The voltage V5 obtained by removing noise from the voltage V4 with the low-pass filter 34 is the output of the processor 30 connected to the flow rate sensing apparatus 20.

Meanwhile, the heat-generating element 23 is driven so as to repeat heat generation and heat dissipation at a constant frequency. For example, the element repeats, about two or three times, a process taking 20 seconds per one cycle including 16 seconds of heat generation and 4 seconds of heat dissipation. By changing the current that flows through the heat-generating element 23 for the heat generation period and the heat dissipation period, both sufficient heat generation and accurate temperature measurement with ignorable influence of self-heating can be performed. That is, the temperature measurement of the drug solution using the heat-generating element 23 is performed during the heat dissipation period.

By repeating the heat generation and heat dissipation in this way, excessive heating of the drug solution is prevented. To be specific, the processor 30 performs control to generate heat so as to keep the temperature rise due to heat generation within 2 [° C.] and so as to stop the heat generation when the temperature rise likely to exceed.

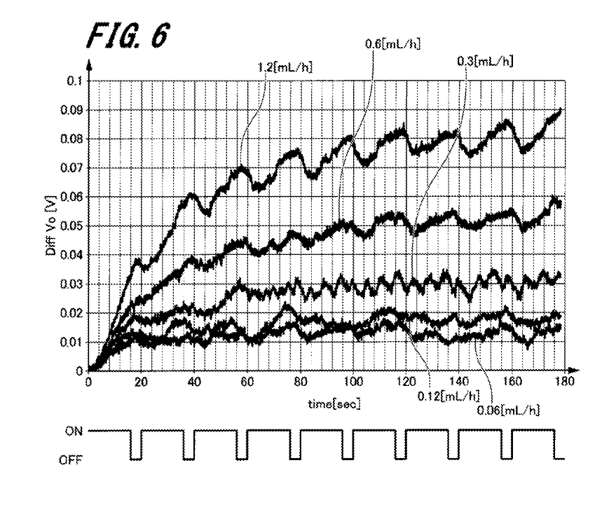

FIG. 6 shows a time variation in the output voltage V5 of the processor 30 when the heat-generating element 23 is controlled in this way. In FIG. 6, the vertical axis represents the value of the voltage V5 and the horizontal axis represents time (sec.). Further, the waveform showing the ON-OFF time variation on the lower side of FIG. 6 shows the timing of turning the heat-generating element 23 on and off. In this example, a process of 20 seconds per one cycle including 16 seconds of heat generation (ON) and 4 seconds of heat dissipation (OFF) is repeated.

FIG. 6 shows examples including five steps set to 0.06 [mL/h], 0.12 [mL/h], 0.3 [mL/h], 0.6 [mL/h], and 1.2 [mL/h], as the flow rates of the pumping apparatus 13. From FIG. 6, it can be seen that the output voltage V5 increases as the flow rate increases.

The characteristic shown in FIG. 6 changes depending on the temperature of the liquid (drug solution). Also, since measurement time cannot be taken too long practically, the flow rate is determined based on the average value in the second cycle zone (20 sec. to 40 sec.) or the average value in the third cycle zone (40 sec. to 60 sec.) in the present embodiment.

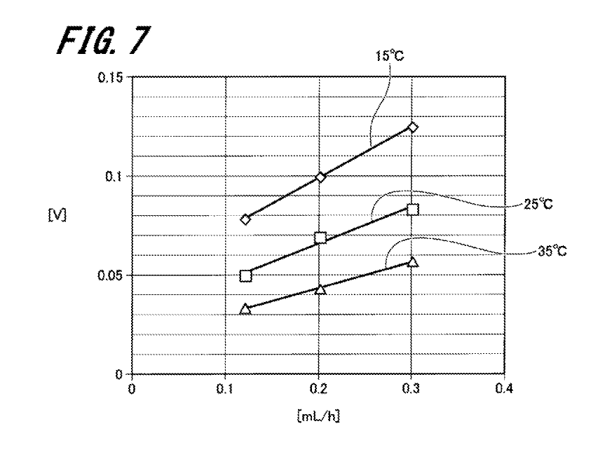

FIG. 7 is a graph that shows plotting of the average voltage value (vertical axis) in the second cycle period (or third cycle period) with respect to the flow rate (horizontal axis). In FIG. 7, examples for three temperatures of 15° C., 25° C., and 35° C. are shown. As shown in FIG. 7, it can be seen that the detected voltage varies in a substantially linear manner for each liquid temperature.

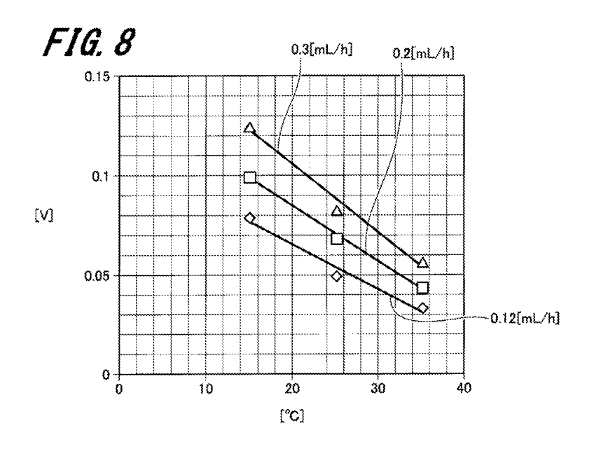

FIG. 8 is a graph showing the relationship between the liquid temperature and the average value in the third cycle zone (40 sec. to 60 sec.) for each flow rate, by using the same data as in FIG. 7. In FIG. 8, the vertical axis represents the average voltage value and the horizontal axis represents the temperature. Here, examples for three flow rates of 0.12 [mL/h], 0.2 [mL/h], and 0.3 [mL/h] are shown.

As shown in FIG. 8, it is understood that the average value of the output voltage V5 varies in a substantially linear manner with respect to the temperature for each flow rate.

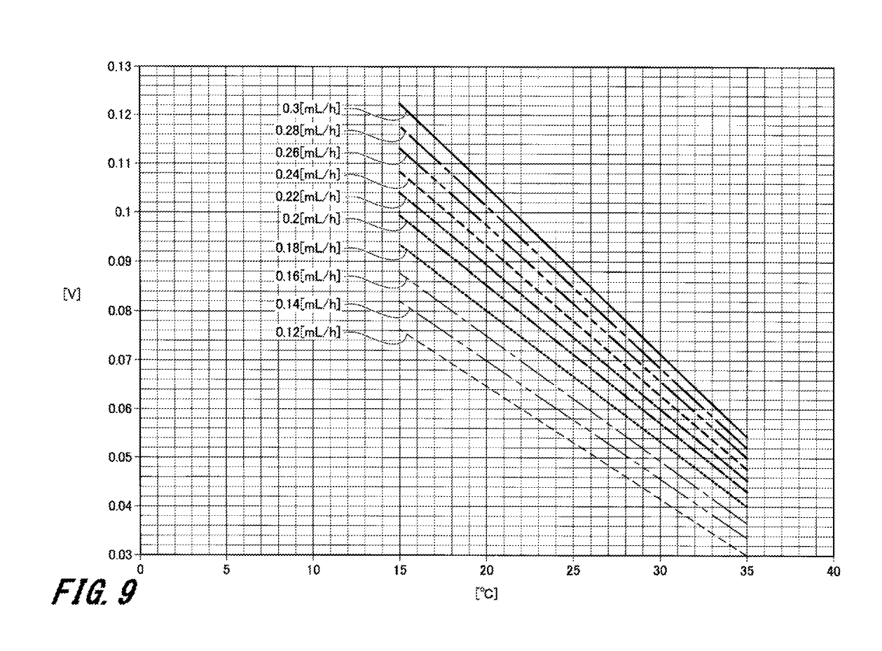

FIG. 9 shows the relationship between the average voltage value (vertical axis) and the temperature of a liquid (drug solution) (horizontal axis) at every flow rate of finely divided 10 levels (maximum 0.3 [mL/h], minimum 0.12 [mL/h]). From the relationship shown in FIG. 9, it is understood that the flow rate can be obtained from the liquid temperature and the average value of the output voltage V5.

By detecting the flow rate in the flow rate sensing apparatus 20 in this way, a flow rate of about 100 [μL/h] at the minimum can be accurately detected.

### 6. Control Example of Heat-Generating Element

It is desirable for the heat-generating element 23 to generate heat with a constant power regardless of the liquid temperature, but such heating cannot be simply performed in the case of using a thermistor. This is because the thermistor has a characteristic in which the resistance value Rt1 thereof varies depending on the liquid temperature.

In the present embodiment, the following method is used so that heat generation with constant power can be performed regardless of the liquid temperature.

Assuming that the power to be given to the thermistor is Pt, the current It satisfies

It=√(Pt/Rt1) (7)

When this current It is given, the voltage Vin to be input is as follows based on the resistance Rin.

Vin=It·Rin

=√V(Pt/Rt1)·Rin (8)

That is, the input voltage Vin may be given by using the equation (8) with respect to the resistance value Rt1 which changes depending on the liquid temperature. In the equation (8), Pt and Rin are constants.

As a method for determining the resistance value Rt1 of the heat-generating element 23, any of the following processings (a) and (b) can be applied, for example.

(a) Processing example of using the resistance value measured during the heat dissipation period of the heat-generating element 23 as the resistance value Rt1 as it is
(b) Processing example of obtaining the resistance value of the heat-generating element 23 from the resistance value of the upstream-side temperature sensing element 22 or the downstream-side temperature sensing element 24 or the temperature information detected from the resistance value

To be specific, the equation (8) is a function of Rt1, and the resistance value Rt1 of the heat-generating element 23 is obtained as a voltage by the above-described processing (a) or (b), so that voltage is referred to as V6. Then, the input voltage Vin with respect to the voltage V6 is held in advance as a table, the input voltage Vin is determined from the obtained voltage V6, and the input voltage Vin is supplied to the terminal 36 (FIG. 4). In the case of the processing (b), when the heat-generating element 23, the upstream-side temperature sensing element 22 and the downstream-side temperature sensing element 24 are all thermistors, the resistance value Rt1 can be obtained directly from the outputs of the upstream-side temperature sensing element 22 and the downstream-side temperature sensing element 24, which is convenient.

### 7. Example of Administration of Drug Solution

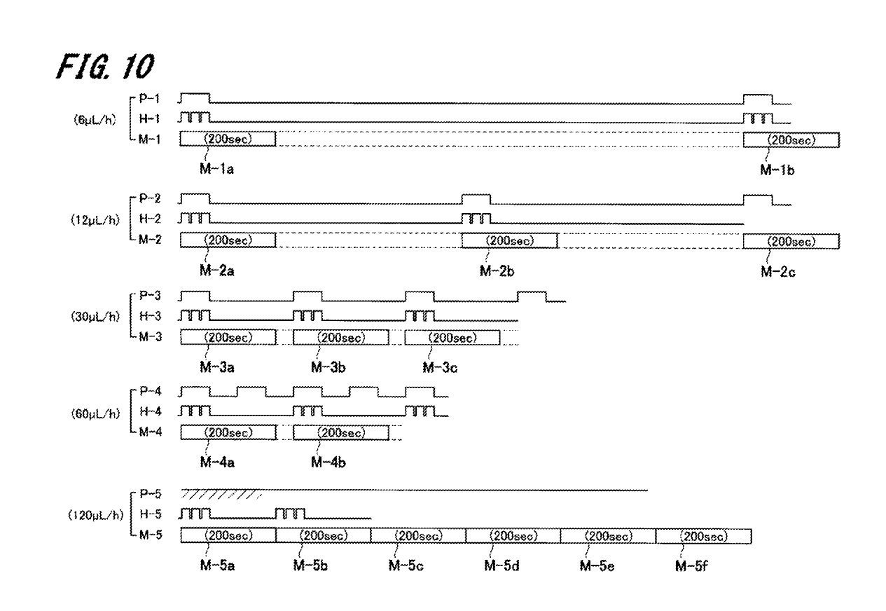

Next, processing in which the drive unit 40 controls the pumping apparatus 13 to administer the drug solution (insulin) to a patient at a low flow rate in conjunction with the flow rate detected by the flow rate sensing apparatus 20 described above will be described with reference to FIG. 10 and Table 1.

As described above, the flow rate sensing apparatus 20 of the present embodiment can detect a flow rate of about 100 [μL/h] at the minimum, but the minimum flow rate required for the drug-solution administration device 10 for administering insulin to a patient is about 0.5 [μL/h], which is even smaller than the minimum flow rate detected amount. When such a very low flow rate is required, the drive unit 40 achieves the required low flow rate by driving the pumping apparatus 13 intermittently.

Here, modes when insulin is administered to a patient will be described. As the modes for administering insulin to a patient, there are a basal insulin administration mode and a bolus insulin administration mode.

The basal insulin administration mode is a mode of continuously administering insulin to a patient at a very low flow rate and the insulin is administered at a very low flow rate of about 0.5 [μL/h] at the minimum.

The bolus insulin administration mode is a mode of temporarily administering insulin to a patient at a high flow rate for the purpose of controlling blood glucose level and the like after each meal, and in accordance with the amount of meal, particularly the amount of carbohydrates contained in food, insulin is administered at a flow rate of several dozen [μL/h] to about 120 [μL/h] in a short period of time.

In order to cope with each of these modes, the drug-solution administration device 10 for administering insulin is required to be capable of variably setting the amount of the drug solution administered within the range of 0.5 [μL/h] to 120 [μL/h].

Further, when starting to use the drug-solution administration device 10, a priming operation of filling the conduit line 17 with insulin is required, and a flow rate of about 300 [μL/h] is necessary in order to perform this operation in a short time.

To be specific, as the drug-solution administration device 10, it is requested that the flow rate can be set in steps of 0.5 [μL/h] in a range from a minimum flow rate of 0.5 [μL/h] to a maximum flow rate of 300 [μL/h]. Accordingly, when a flow rate equal to or lower than the flow rate that can be detected by the flow rate sensing apparatus 20 is required, the drug-solution administration device 10 of the present embodiment intermittently administers the drug solution by the pumping apparatus 13, whereby a necessary amount of drug solution is administered.

Here, as an actual operation example of the pumping apparatus 13, the following three patterns are prepared.

Pattern 1: (when the Required Flow Rate is 0.5 [μL/h])

The intermittent driving in which the pumping apparatus 13 feeds the liquid at 90 [μL/h] for 40 seconds and then pauses for 119 minutes and 20 seconds is repeated.

Pattern 2: (when the Required Flow Rate is in the Range of 1 [μL/h] to 119.5 [μL/h])

The pumping apparatus 13 repeats intermittent driving in which the pumping apparatus 13 is paused for time variably set within a range of 119 minutes to 12 seconds in accordance with the flow rate after pumping at 120 [μL/h] for 60 seconds (see Table 1).

Pattern 3: (when the Required Flow Rate is in the Range of 120 [μL/h] to 300 [μL/h])

The drive voltage and the drive frequency of the pumping apparatus 13 are changed to perform continuous driving.

The following Table 1 shows a setting example of downtime (minutes) at the required flow rate 1 [μL/h] to 119.5 [μL/h] in the case of pattern 2. In this example of Table 1, the liquid feeding is performed at 120 [μL/h] for 60 seconds (1 minute) at any required flow rate, and the required flow rate is set by adjusting the downtime after the liquid delivery.

TABLE 1
Set flow rate (μL/h)/liquid delivery time (minutes)/downtime
(minutes)

1/1/119
2/1/59
3/1/39
4/1/29
5/1/23
6/1/19
8/1/14
9/1/12.333
10/1/11
12/1/9
15/1/7
16/1/6.5
18/1/5.666
20/1/5
24/1/4
25/1/3.8
30/1/3
40/1/2
50/1/1.4
60/1/1
80/1/0.5
90/1/0.333
100/1/0.2
120/1/0

The [Table 1] shows that [1/1/119] in the uppermost column, for example, is achieved by repeating one minute delivery at 120 [μL/h] and 119 minutes pause when the set flow rate is 1 [μL/h]. The last column [120/1/0] shows one minute delivery at 120 [μL/h] and 0 minutes pause when the set flow rate is 120 [μL/h], in other words, a set flow rate of 120 [μL/h] is achieved by continuous delivery without pause.

In the case of driving by intermittent operation, the flow rate sensing apparatus 20 measures the flow rate during a period of liquid feeding of 40 seconds or 1 minute, and when the flow rate measured value that is obtained is higher than the target value, the drive unit 40 set the downtime to be longer than the set time (for example, the time shown in Table 1). Conversely, when the obtained flow rate measured value is lower than the target value, the drive unit 40 reduces the downtime to less than the set time (for example, the time shown in Table 1). By performing such control, very high-accuracy flow rate control having ±5% flow rate accuracy becomes possible.

FIG. 10 is a diagram showing setting examples of the driving periods P-1, P-2, P-3, P-4, and P-5 of the pumping apparatus 13, the heat generation periods H-1, H-2, H-3, H-4, and H-5 of the heat-generating element 23 (heater) in the flow rate sensing apparatus 20, and measurement periods M-1, M-2, M-3, M-4, and M-5 of the flow rate sensing apparatus 20.

The five examples shown in FIG. 10 are examples at flow rates of 6 [μL/h], 12 [μL/h], 30 [μL/h], 60 [μL/h], and 120 [μL/h], respectively.

The respective measurement periods of 200 seconds shown in FIG. 10 are detecting periods of one cycle in which the flow rate sensing apparatus 20 detects the flow rate.

In principle, it is necessary for the flow rate sensing apparatus 20 of the present embodiment to wait until the influence of heat generation disappears. For this reason, a 200 second process is performed in which a 20 second process including heat generation for 16 seconds and heat releasing for 4 seconds (suspension of heat generation) thereafter is repeated three times and then heat is released for further 140 seconds for example. The flow rate sensing apparatus 20 periodically detects the flow rate with the period of 200 seconds as one cycle. Flow rate detection for one cycle of 200 seconds is performed intermittently or continuously for each flow rate as shown in FIG. 10.

Here, as shown in FIG. 10, intermittent driving of the pumping apparatus 13 is required depending on the target flow rate. For example, when the flow rate is 30 [μL/h] or less, the pumping apparatus 13 is driven for the first 60 seconds of the 200 seconds for which the flow rate is detected.

For example, when the flow rate is 6 [μL/h], the driving period P-1 of the pumping apparatus 13 and the heat generation period H-1 of the heat-generating element 23 are set for intermittent operation in conjunction with each other, and measurement periods M-1a, M-1b, . . . of 200 seconds are set in conjunction with each start of the driving period P-1 and the heat generation period H-1.

When the flow rate is 12 [μL/h], the driving period P-2 of the pumping apparatus 13 and the heat generation period H-2 of the heat-generating element 23 are set for intermittent operation at shorter intervals than at the flow rate of 6 [μL/h]. Then, measurement periods M-2a, M-2b, M-2c, . . . of 200 seconds are set in conjunction with each start of the driving period P-2 and the heat generation period H-2.

When the flow rate is 30 [μL/h], the driving period P-3 of the pumping apparatus 13 and the heat generation period H-3 of the heat-generating element 23 are set for intermittent operation at further shorter intervals. Then, measurement periods M-3a, M-3b, M-3c, . . . of 200 seconds are set in conjunction with each start of the driving period P-3 and the heat generation period H-3.

When the flow rate is 60 [μL/h], a cycle of 120 seconds is set for repeating driving and stopping of the pumping apparatus 13 every 60 seconds.

That is, when the flow rate is 60 [μL/h], a cycle of 120 seconds is set, in which the driving period P-4 of the pumping apparatus 13 is repeated every 60 seconds. The heat generation period H-4 is set for operation once every two driving periods P-4 of the pumping apparatus 13. Then, measurement periods M-4a, M-4b, . . . of 200 seconds are set in conjunction with each start of the heat generation periods H-4.

Further, when the flow rate is 120 [μL/h], the pumping apparatus 13 is continuously driven.

In other words, when the flowrate is 120 [μL/h], the driving period P-5 of the pumping apparatus 13 is set continuous. The heat generation period H-5 of the heat-generating element 23 is set intermittent at intervals of 200 seconds. Then, measurement periods of 200 seconds M-5a, M-5b, M-5c, M-5d, M-5e, M-5f, . . . are set continuous.

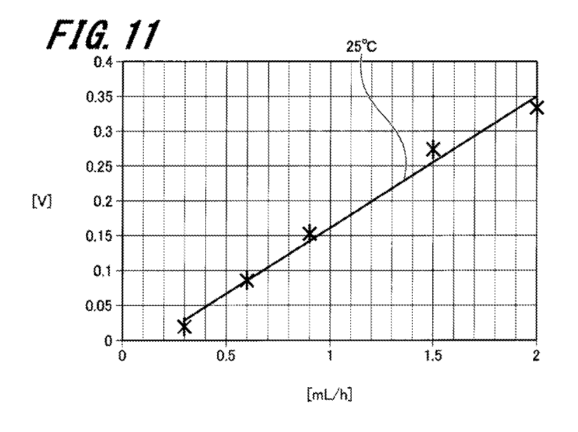

FIG. 11 shows the relationship between the voltage V5 (vertical axis) detected by the flow rate sensing apparatus 20 and the flow rate (horizontal axis) in the case of a liquid temperature of 25° C.

As shown in FIG. 11, it becomes possible to detect the flow rate in detail based on the voltage V5 and to perform control to bring the flow rate closer to the target flow rate.

### 8. Another Configuration Example of the Flow Rate Sensing Apparatus

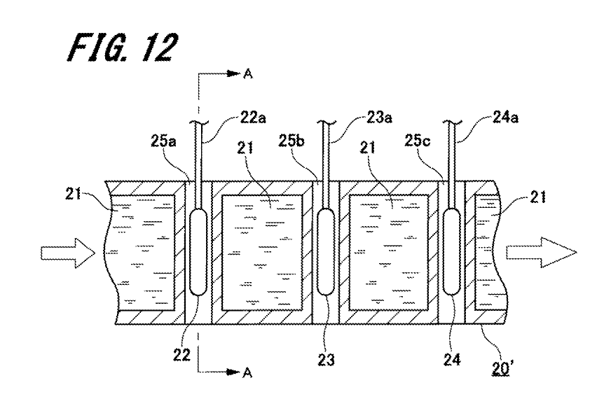

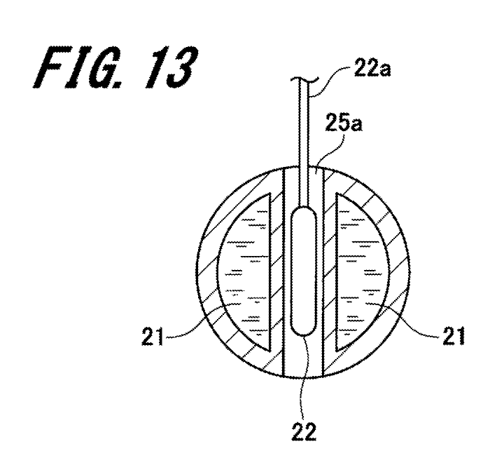

FIGS. 12 and 13 show a flow rate sensing apparatus 20′ having a structure different from that of the flow rate sensing apparatus 20 shown in FIG. 2.

The flow rate sensing apparatus 20′ shown in FIG. 12 is provided with tubular portions 25a, 25b, and 25c at equal intervals in the conduit line 21. The drug solution does not enter the tubular portions 25a, 25b, and, 25c and a drug solution is allowed to pass through the peripheries of the tubular portions 25a, 25b, and 25c as shown in FIG. 13. FIG. 13 shows a cross section of the portion where the tubular portion 25a is disposed, but the cross sections of the portions where the other tubular portions 25b and 25c are disposed have the same configuration. Each of the tubular portions 25a, 25b, and 25c is made of a material having high thermal conductivity.

Then, the first temperature sensing element (upstream-side temperature sensing element) 22 is disposed in the upstream-side tubular portion 25a, the heat-generating element 23 is disposed in the central tubular portion 25b, and a second temperature sensing element (downstream-side temperature sensing element) 24 is disposed in the downstream-side tubular portion 25c.

Although not shown in FIGS. 12 and 13, a material such as a cream for improving thermal bonding is disposed between the tubular portions 25a, 25b, and 25c and the elements 22, 23, and 24, respectively.

The circuits connected to the elements 22, 23, and 24 are the same as those in the example of FIGS. 3 and 4.

According to the flow rate sensing apparatus 20′ configured as described above, since the elements 22, 23, and 24 are isolated from the passage of the drug solution as compared with the flow rate sensing apparatus 20 shown in FIG. 2, eluate entering the drug solution passing through the conduit line 21 can be reduced.

### 9. Example of Improving Flow Rate Sensing Accuracy

Next, an example of further improving the flow rate sensing accuracy when the processor 30 detects the flow rate from the output voltage of the flow rate sensing apparatus 20 will be described.

For example, the flow rate is very low at the time of basal insulin administration in which insulin is administered to a patient at a low flow rate, so that the output voltage detected by the processor 30 is easily affected by an offset and drift. Processing for improving the flow rate sensing accuracy even in a situation where such an offset or drift is affecting will be described.

As already described with reference to FIG. 11 and the like, the output of the processor 30 changes linearly with respect to each flow rate and shows a high correlation coefficient. In addition, the gradient of the approximate straight line shown in each figure changes linearly with respect to temperature and exhibits a high correlation coefficient.

Accordingly, a flow rate with high sensing accuracy can be obtained from the output voltage of the processor 30 by the following procedures (a), (b) and (c).

(a) When the flow rate is referred to as x and the output of the processor 30 is referred to as y, the following characteristic curve is obtained in advance for each temperature.

y=ax+b (9)

a is a gradient for each temperature.

Further, regarding the obtained gradient a for each temperature, the following relational expression with the temperature T is obtained.

a=αT+_{R} (10)

Here, α and β are constants.

(b) When the flow rate is zero before (or after) the flow rate measurement, the output y of the processor 30 is measured to find the y-intercept b(T) at the temperature at the measurement time. Then, from the data of the gradient a(T) acquired in advance, the following characteristic curve at the temperature at the measurement time is determined.

y=a(T)x+b(T) (11)

(c) Calculate the flow rate x from the measured circuit output y and equation (11).

By obtaining the flow rate x in this way, the flow rate that excludes the influence of the offset and drift can be detected with high precision.

### 10. Modification Example

It is to be noted that the configuration described in the above embodiment is a preferable example, and the present invention is not limited to the configuration described in the embodiment. For example, a piezoelectric pump is used as the pumping apparatus, but a pump of other configuration may be used.

As shown in FIG. 2, with respect to each of the elements 22, 23, and 24 arranged in the conduit line 21 constituting the flow rate sensing apparatus 20, some coating may be applied to the surface to reduce the amount of eluate entering a drug solution passing through the conduit line 21.

Further, in the above-described embodiment, the measurement of the liquid temperature in the conduit line is performed by the heat-generating element itself by utilizing the non-heat generating period of the heat-generating element. In contrast, a dedicated temperature sensing element for measuring the liquid temperature inside the conduit line may be provided.

For example, liquid temperature may be measured by disposing an element (such as a thermistor) for measuring the liquid temperature further upstream of the first temperature sensing element 22 shown in FIG. 2 or 12 to reduce the influence of heat generation by the heat-generating element. By doing this, it becomes possible to conduct administration at lower flow rate with higher accuracy.

Further, in the above-described embodiment, although the case of application to the portable insulin administration device has been described, embodiments described herein may be applied to a drug-solution administration device for administering drug solutions other than insulin. In addition, a configuration as a portable type in a small size is one example, and the configuration may be a stationary type.

### REFERENCE NUMERAL LIST

10 drug-solution administration device
11 reservoir
12, 14, 16, 17, 21 conduit line
13 pumping apparatus
15 microneedle valve
20, 20′ flow rate sensing apparatus
21a, 21b, 21c element disposing position
22 first temperature sensing element (upstream-side temperature sensing element)
22a terminal
23 heat-generating element
23a terminal
24 second temperature sensing element (downstream-side temperature sensing element)
24a terminal
25a, 25b, 25c tubular portion
30 processor
31 first voltage conversion circuit
32 second voltage conversion circuit
33 differential amplifier circuit
34 low-pass filter
31a, 32a, 33a, 34a operational amplifier
35 output terminal
36 terminal
37 operational amplifier
38 terminal
40 drive unit
C1 capacitor
P1 characteristic of flow rate of a single unit
P2 fluctuation of flow rate at output portion
R1, R2, R3, R5, R6, R8, R9, R12, R14, R17, R19, R21, Rin resistor

What is claimed is:

1. A device for administering a drug solution, the device comprising:
a pumping apparatus configured to deliver a drug solution held in a reservoir to a live body;
a flow rate sensing apparatus configured to determine a flow rate of the solution delivered by the pumping apparatus, the flow rate sensing apparatus comprising:
a heat-generating element disposed in a conduit line in which the drug solution is delivered;
a first temperature sensor disposed in the conduit line on an upstream side of the heat-generating element;
a second temperature sensor disposed in the conduit line on a downstream side of the heat-generating element; and
an processor configured to calculate the flow rate in the conduit line based on a difference between a temperature detected by the first temperature sensing element and a temperature detected by the second temperature sensing element; and
a controller configured to control a drive state of the pumping apparatus based on the flow rate determined by the flow rate sensing apparatus.

2. The device according to claim 1, wherein:
the heat-generating element is configured to alternately operate in a heat generation state and a non-heat generation state for a fixed period of time, and
the processor is configured to calculate the flow rate during the fixed period from an average value of the difference between the temperature detected by the first temperature sensing element and the temperature detected by the second temperature sensing element during the fixed period.

3. The device according to claim 2, wherein:
the heat-generating element is configured to generate heat by application of a voltage, and has a resistance value that varies with temperature, and
the flow rate sensing apparatus is configured to prevent the drug solution from rising to a temperature higher than a threshold value based on temperature information detected from the resistance value of the heat-generating element during the non-heat generation state.

4. The device according to claim 3, wherein:
the heat-generating element is configured to generate heat using constant power during a period when the heat-generating element is in the heat generation state using temperature information detected by the first temperature sensing element and the second temperature sensing element or temperature information detected from the resistance value of the heat-generating element.

5. The device according to claim 1, further comprising:
a third temperature sensing element on an upstream side of the first temperature sensing element.

6. The device according to claim 1, wherein:
the pumping apparatus comprises a piezoelectric pump that comprises a piezoelectric element, and
the controller is configured to control the drive state of the pumping apparatus by setting a driving voltage and a driving frequency at which the piezoelectric element is driven.

7. The device according to claim 1, wherein:
a distance between the first temperature sensor and the heat-generating element is equal to a distance between the second temperature sensor and the heat-generating element.

8. The device according to claim 1, wherein:
the heat-generating element, the first temperature sensor, and the second temperature sensor are disposed in the conduit line and provided with a coating adapted to reduce an amount of eluate entering a drug solution passing through the conduit line.

9. The device according to claim 1, wherein:
the heat-generating element, the first temperature sensor, and the second temperature sensor are disposed in contact with an area through which a drug solution passes in the conduit line via a partition wall.

10. A method for administering a drug solution, the method comprising:
providing a device comprising:
a pumping apparatus configured to deliver a drug solution held in a reservoir to a live body,
a flow rate sensing apparatus configured to determine a flow rate of the solution delivered by the pumping apparatus, the flow rate sensing apparatus comprising:
a heat-generating element disposed in a conduit line in which the drug solution is delivered,
a first temperature sensor disposed in the conduit line on an upstream side of the heat-generating element, and
a second temperature sensor disposed in the conduit line on a downstream side of the heat-generating element;
heating the drug solution in the conduit line with the heat-generating element;
calculating the flow rate in the conduit line based on a difference between a temperature detected by the first temperature sensing element and a temperature detected by the second temperature sensing element; and
controlling a drive state of the pumping apparatus based on the flow rate determined by the flow rate sensing apparatus.

11. The method according to claim 10, wherein:
the step of heating the drug solution comprises alternately operating the heat-generating element in a heat generation state and a non-heat generation state for a fixed period of time, and
calculating the flow rate during the fixed period from an average value of the difference between the temperature detected by the first temperature sensing element and the temperature detected by the second temperature sensing element during the fixed period.

12. The method according to claim 11, wherein:
the heat-generating element is configured to generate heat by application of a voltage, and has a resistance value that varies with temperature, and
the flow rate sensing apparatus is configured to prevent the drug solution from rising to a temperature higher than a threshold value based on temperature information detected from the resistance value of the heat-generating element during the non-heat generation state.

13. The method according to claim 12, wherein:
the heat-generating element is configured to generate heat using constant power during a period when the heat-generating element is in the heat generation state using temperature information detected by the first temperature sensing element and the second temperature sensing element or temperature information detected from the resistance value of the heat-generating element.

14. The method according to claim 10, wherein the device further comprises:
a third temperature sensing element on an upstream side of the first temperature sensing element.

15. The method according to claim 10, wherein:
the pumping apparatus comprises a piezoelectric pump that comprises a piezoelectric element, and
the controller is configured to control the drive state of the pumping apparatus by setting a driving voltage and a driving frequency at which the piezoelectric element is driven.

16. The method according to claim 10, wherein:
a distance between the first temperature sensor and the heat-generating element is equal to a distance between the second temperature sensor and the heat-generating element.

17. The method according to claim 10, wherein:
the heat-generating element, the first temperature sensor, and the second temperature sensor are disposed in the conduit line and provided with a coating adapted to reduce an amount of eluate entering a drug solution passing through the conduit line.

18. The method according to claim 10, wherein:
the heat-generating element, the first temperature sensor, and the second temperature sensor are disposed in contact with an area through which a drug solution passes in the conduit line via a partition wall.
